(19) United States
(12) Patent Application Publication
(10) Pub. No.: US 2024/0245627 A1
(43) Pub. Date: Jul. 25, 2024

(54) ALKENE QUINONE COMPOUNDS AND METHODS OF USE

(71) Applicant: University of Pittsburgh - Of the Commonwealth System of Higher Education, Pittsburgh, PA (US)

(72) Inventors: Adam C. Straub, Pittsburgh, PA (US); Bruce A. Freeman, Pittsburgh, PA (US); Francisco J. Schopfer, Pittsburgh, PA (US)

(73) Assignee: University of Pittsburgh - Of the Commonwealth System of Higher Education, Pittsburgh, PA (US)

(21) Appl. No.: 18/282,099

(22) PCT Filed: Mar. 24, 2022
(86) PCT No.: PCT/US2022/021714

§ 371 Date: Sep. 14, 2023

Related U.S. Application Data
(60) Provisional application No. 63/166,058, filed on Mar. 25, 2021.

(51) Int. Cl. A61K 31/122; A61P 29/00; C07C 205/46

(52) U.S. Cl. CPC A61K 31/122; A61P 29/00; C07C 205/46; C07B 2200/05

(57) ABSTRACT

Disclosed herein are new pharmacological therapies that increase CYB5R3 activity, improve vascular function and limit inflammation by restoring the redox balance in CVD. In one embodiment, disclosed herein are compounds that include an alkyl chain containing a nitroalkene group conjugated to a quinone-containing moiety.

This application claims the benefit of U.S. Provisional Appl. No. 63/166,058, filed on Mar. 25, 2021, which application is incorporated herein by reference.

### ACKNOWLEDGMENT OF GOVERNMENT SUPPORT

This invention was made with government support under grants R01HL128304, R01HL133864, R01HL14825, P01HL103455, R01HL132550, R01DK112854, and R01GM125944 awarded by the National Institutes of Health. The government has certain rights in the invention.

### BACKGROUND

Statistics from the American Heart Association show that 1 of every 3 deaths in the US is linked to cardiovascular disease (CVD) costing approximately $351.2 billion in expenditures. A common element to the initiation and progression of cardiovascular disease is an imbalance of reduction and oxidation (redox) signaling, defined as the specific, usually reversible, redox modification of cellular signaling pathway components by oxidants (i.e. superoxide, hydrogen peroxide, and downstream products of oxidant-nitrogen oxide reactions). In the heart and blood vessels, redox signaling regulates several physiological processes (e.g., excitation-contraction coupling, vasodilation) and is involved in a wide array of pathophysiological and homoeostatic or stress response pathways.

Oxidants involved in cardiovascular redox signaling may derive from many sources including NADPH oxidases, xanthine oxidase, mitochondria electron transport chain uncoupling, nitric oxide (NO) synthases and a panoply of other metalloproteins. Although FDA approved drugs such as blood pressure and cholesterol lowering agents have shown some therapeutic benefits in patients, it is apparent that many patients continue to suffer from progressive CVD, allied end-organ dysfunction and significant mortality, indicating that additional novel and efficacious therapies are needed and new drug targets should be identified. NADH cytochrome b5 reductase 3 (CYB5R3) is a key enzyme that maintains redox balance. CYB5R3, otherwise known as methemoglobin reductase, is a flavoprotein recognized for its ability to transfer electrons from its NADH domain through cytochrome b5 (CYB5) to an electron acceptor. CYB5R3 is broadly located in vascular and other cells; on the cytosolic face of the outer mitochondrial membrane, endoplasmic reticulum and plasma membrane. Membrane-bound CYB5R3 regulates several functionally-significant biological reactions including heme reduction, elongation and desaturation of fatty acids, Co-enzyme Q (CoQ) reduction, cholesterol biosynthesis, and drug metabolism in the liver. The soluble, spliced (loss of 33 amino acids on the N-terminus) form of CYB5R3 reduces erythrocytic methemoglobin. In the human population, over 40 genetic polymorphisms in CYB5R3 have been identified. Of particular interest is the missense variant at position 117 of the soluble protein, equivalent to position 150 of membrane CYB5R3, wherein the amino acid threonine is substituted for a serine residue (variant #rs1800457). This is a high frequency genetic variant in individuals with African Ancestry (23% minor allele frequency), with this a population that endures a high burden of CVD. This genetic variant is found in less than 1% of Caucasians. The functions of CYB5R3 in the cardiovascular system are physiologically significant. CYB5R3 regulates the redox state of hemoglobin a in small artery and arteriolar endothelial cells, controlling NO diffusion to VSMC. CYB5R3 sensitizes soluble guanylate cyclase (sGC) to its physiological ligand NO by reducing sGC heme iron, thus functioning as a control mechanism for intracellular cGMP levels in vascular smooth muscle cells. Conditional deletion of CYB5R3 in adult cardiomyocytes increases oxidative stress, decreases CoQ levels, promotes heme iron oxidation and decreases NO signaling, affirming that CYB5R3 expression and activity is critical for cardiac health.

### SUMMARY

Disclosed herein is a compound, or a pharmaceutically acceptable salt thereof, of:

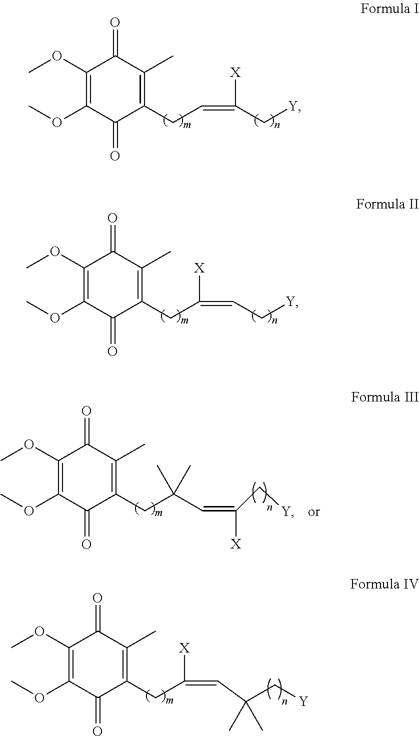

wherein X is an electron withdrawing group;
Y is a terminal group that inhibits enzymatic omega-end hydroxylation, oxidation and shortening of the alkyl chain to which Y is attached, or Y is H;
m is 1 to 10; and
n is 3 to 15.

Disclosed herein is a compound, or a pharmaceutically acceptable salt thereof, of:

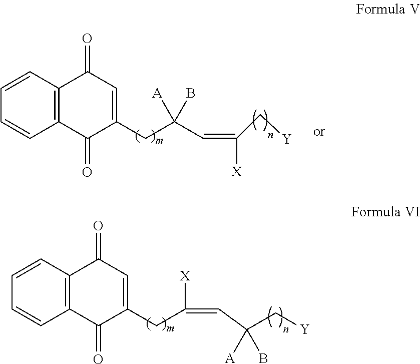

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

Disclosed herein is a compound, or a pharmaceutically acceptable salt thereof, of:

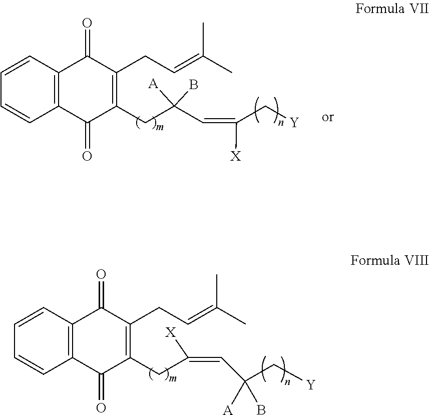

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

Disclosed herein is a compound, or a pharmaceutically acceptable salt thereof, of:

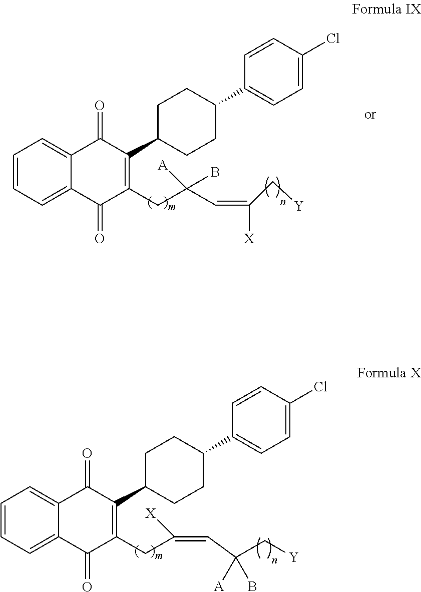

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

Disclosed herein is a compound, or a pharmaceutically acceptable salt thereof, of:

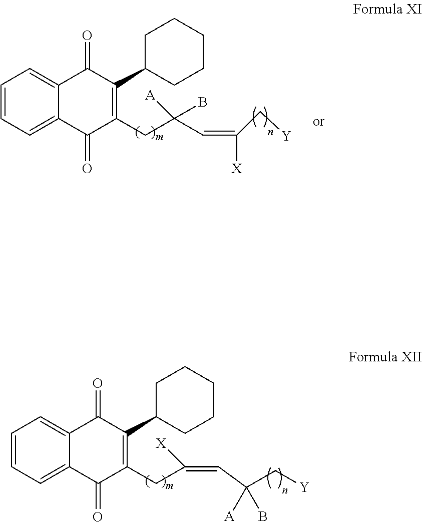

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

Disclosed herein is a compound, or a pharmaceutically acceptable salt thereof, of:

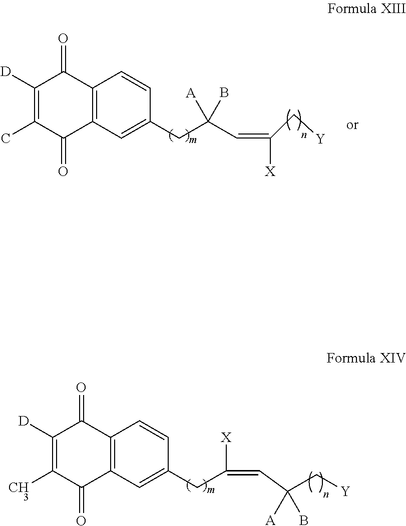

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

Disclosed herein is a compound, or a pharmaceutically acceptable salt thereof, of:

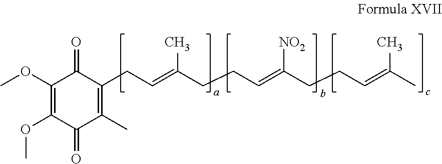

wherein a is 0 to 9, b is 1, and c is 0 to 9.

Disclosed herein is a compound, or a pharmaceutically acceptable salt thereof, of:

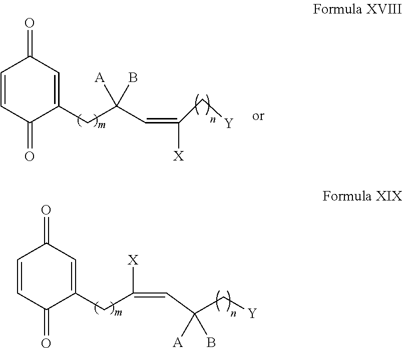

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

Disclosed herein is a compound, or a pharmaceutically acceptable salt thereof, of:

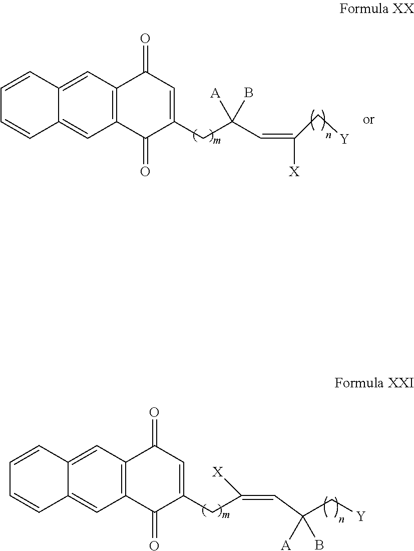

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

Also disclosed herein is a method comprising administering a therapeutically effective amount of a compound disclosed herein to a subject having, suspected of having, or at risk of developing, cardiovascular disease.

Also disclosed herein is a method of treating cardiovascular disease in a subject, comprising administering a therapeutically effective amount of a compound disclosed herein to the subject, thereby treating the cardiovascular disease.

Also disclosed herein is a method of treating cardiovascular disease in a subject, comprising administering a therapeutically effective amount of compound comprising an alkyl chain containing a nitroalkene group, wherein the alkyl chain containing a nitroalkene group is conjugated to a quinone-containing moiety, thereby treating the cardiovascular disease.

Also disclosed herein is a method of treating a disease or condition associated with a redox imbalance in a subject, comprising administering a therapeutically effective amount of a compound disclosed herein to the subject, thereby treating the disease or condition associated with a redox imbalance.

Also disclosed herein is a method comprising administering to a subject a compound disclosed herein, thereby enhancing CYB5R3 activity in the subject.

Also disclosed herein is a method for inhibiting an inflammatory condition or disease associated with pro-inflammatory genes IL-1, VCAM-1, ICAM-1 CCL5, CCL20 or CXCL10 in a subject, comprising administering a therapeutically effective amount of a compound disclosed herein to the subject, thereby treating the inflammatory condition or disease.

Also disclosed herein is a method comprising administering to a subject a compound disclosed herein, thereby decreasing mean arterial blood pressure in the subject.

Also disclosed herein is a method comprising administering to a subject a compound disclosed herein, thereby decreasing pulse pressure in the subject.

Also disclosed herein is a method comprising administering to a subject a compound disclosed herein, thereby reversing endothelial cell dysfunction in the subject.

Also disclosed herein is a method of treating sickle cell disease in a subject, comprising administering a therapeutically effective amount of a compound disclosed herein, thereby treating the sickle cell disease.

Also disclosed herein is a method of treating a disease or condition in a subject, comprising administering a therapeutically effective amount of a compound disclosed herein to the subject, thereby treating the disease or condition, wherein the disease or condition is arterial stiffness, systemic and pulmonary hypertension, sickle cell disease, heart failure with reduced ejection fraction, heart failure with preserved ejection fraction, arterial stenosis, vascular aneurysm, obesity, neurodegenerative disorders, skin disorders, arthritis, vasculitis, burns, autoimmune disease, autoinflammatory disease, lupus, Lyme's disease, gout, sepsis, hyperthermia, ulcers, enterocolitis, osteoporosis, viral or bacterial infections, cytomegalovirus, periodontal disease, glomerulonephritis, sarcoidosis, lung disease, chronic lung injury, respiratory distress, lung inflammation, fibrosis of the lung, asthma, acquired respiratory distress syndrome, tobacco induced lung disease, granuloma formation, fibrosis of the liver, graft vs. host disease, postsurgical inflammation, coronary and peripheral vessel restenosis following angioplasty, stent placement or bypass graft, acute and chronic leukemia, B lymphocyte leukemia, neoplastic diseases, arteriosclerosis, atherosclerosis, myocardial inflammation and fibrosis, psoriasis, immunodeficiency, disseminated intravascular coagulation, systemic sclerosis, amyotrophic lateral sclerosis, multiple sclerosis, Parkinson's disease, Alzheimer's disease, encephalomyelitis, edema, inflammatory bowel disease, hyper IgE syndrome, cancer metastasis or growth, adoptive immune therapy, reperfusion syndrome, radiation burns, or alopecia.

The foregoing will become more apparent from the following detailed description, which proceeds with reference to the accompanying figures.

### BRIEF DESCRIPTION OF THE DRAWINGS

FIG. 1. Conceptual design of quinone-linked nitro alkene (Q-NA), a novel small molecule that is designed to reverse heme oxidation, scavenge oxidants, protect thiols from oxidation, inhibit pro-inflammatory mediator generation and activate tissue-protective gene expression. These latter two pleiotropic actions occur via inhibition of NFkB-dependent gene expression and activation of Keap1/NRF-2-regulated gene expression. CoQ₀ (pink) acts to reduce inactive heme moieties and limit reactive oxygen species formation. Conjugation of CoQ₀ to a nitroalkene-containing substituent (blue) will enable upregulation of antioxidant and repair genes via NRF-2 activation, inhibition of pro-inflammatory genes via NFkB inhibition and block thiols from oxidation through cysteine adduction. The pleiotropic actions of Q-NA are intended to restore cell and organ homeostasis in diseases associated with a redox imbalance.

FIGS. 2a-2c. Reduction of oxidized myoglobin by CYB5R3 is not affected by (FIG. 2a) oleic acid or (FIG. 2b) Q-OA (CoQ-linked oleic acid). Q-NA enhanced (FIG. 2c) CYB5R3 reduction of myoglobin. Error bars are S.E.M. and n=3.

FIGS. 3a-3c. FIG. 3a) Schematic diagram showing experimental design. FIG. 3b) mRNA profile of top 50 genes changed based on p-value. FIG. 3c) mRNA expression profile of genes uniquely regulated by Q-NA. (n=5)

FIGS. 4a-4b. Western blot analysis of pro-inflammatory proteins VCAM-1 and ICAM-1 (FIG. 4a) and inflammatory genes IL-6, MCP-1, IL-1beta, VCAM-1, ICAM-1, CCL5, CCL20, and CXCL10 (FIG. 4b) from human aortic endothelial cells treated with vehicle (DMSO), Q-NA (5 μM), lipopolysaccharide (1 μg/mL) and Q-NA (5 μM)+lipopolysaccharide (1 μg/mL). Error bars show S.E.M, p-value calculated by a 1-way ANOVA.

FIGS. 5a-5c. FIG. 5a), Delta change in mean arterial blood pressure in a 2 intact kidney, 1 renal artery clipped murine model of hypertension treated with or without Q-NA. FIG. 5b), Delta change in pulse pressure in 2-kidney, 1 clip mice treated with vehicle or 2 doses of Q-NA. FIG. 5c), Aortic relaxation responses to acetylcholine from control wild type, 2-kidney, 1 clip mice or 2-kidney, 1 clip mice treated with Q-NA. Error bars show S.E.M, p-value calculated by a 1 or 2-way ANOVA.

FIGS. 6a-6d. FIG. 6a. Schematic showing experimental design for transgenic Sickle Cell mouse treatment with Q-NA (25 mg/kg) for two weeks. FIG. 6b. Hematology at of a normal C57B/L6 mice (AA, left column), Sickle Cell mice (SS, middle column) and Sickle Cell mice following 2 weeks of Q-NA treatment (SS+Q-NA). FIGS. 6c and 6d. Ex vivo pulmonary artery relaxation responses of Sickle Cell mice treated for 2 weeks with Q-NA following administration of the endothelium dependent vasodilator acetylcholine and an endothelium-independent vasodilator sodium nitroprusside from.

FIGS. 7a-7e. FIG. 7a. Schematic of modified nitroalkene quinone derivatives. FIGS. 7b-e. Structure-activity relationships comparing Q-NA, BenzoQ-NA, NapthoQ-NA and AnthraQ-NA on heme-oxygenase-1 (Hmox-1), Growth differentiation factor-15 (GDF-15), Kruppel-like factor 2 (KLF-2) and spingosine 1 phosphate receptor 3 (Slpr3) mRNA expression.

FIG. 8. Q-NA requires CYB5R3 to induce RPS/RPL gene induction with and without LPS.

FIG. 9. NO₂—OA and Q-NA stimulate divergent effects of RSP/RPL gene transcription in aortic endothelial cells.

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Administration” as used herein is inclusive of administration by another person to the subject or self-administration by the subject.

“Alkenyl” refers to a cyclic, branched or straight chain group containing only carbon and hydrogen, and contains one or more double bonds that may or may not be conjugated. Alkenyl groups may be unsubstituted or substituted. “Lower alkenyl” groups contain one to six carbon atoms.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

“Alkynyl” refers to a cyclic, branched or straight chain group containing only carbon and hydrogen, and unless otherwise mentioned typically contains one to twelve carbon atoms, and contains one or more triple bonds. Alkynyl groups may be unsubstituted or substituted. “Lower alkynyl” groups are those that contain one to six carbon atoms.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals. Illustrative non-human mammals include animal models (such as mice), non-human primates, companion animals (such as dogs and cats), livestock (such as pigs, sheep, cows), as well as non-domesticated animals, such as the big cats. The term subject applies regardless of the stage in the organism's lifecycle. Thus, the term subject applies to an organism in utero or in ovo, depending on the organism (that is, whether the organism is a mammal or a bird, such as a domesticated or wild fowl).

The term “co-administration” or “co-administering” refers to administration of a compound disclosed herein with at least one other therapeutic agent or therapy within the same general time period, and does not require administration at the same exact moment in time (although co-administration is inclusive of administering at the same exact moment in time). Thus, co-administration may be on the same day or on different days, or in the same week or in different weeks. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent and/or lowers the frequency of administering the potentially harmful (e.g., toxic) agent. “Co-administration” or “co-administering” encompass administration of two or more active agents to a subject so that both the active agents and/or their metabolites are present in the subject at the same time. Co-administration includes simultaneous administration in separate compositions, administration at different times in separate compositions, or administration in a composition in which two or more active agents are present.

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's lifecycle. Thus, the term subject applies to an organism in utero or in ovo, depending on the organism (that is, whether the organism is a mammal or a bird, such as a domesticated or wild fowl).

“Substituted” or “substitution” refers to replacement of a hydrogen atom of a molecule or an R-group with one or more additional R-groups. Unless otherwise defined, the term “optionally-substituted” or “optional substituent” as used herein refers to a group which may or may not be further substituted with 1, 2, 3, 4 or more groups, preferably 1, 2 or 3, more preferably 1 or 2 groups. The substituents may be selected, for example, from C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆ alkoxyaryl, halo, C₁₋₆alkylhalo (such as CF₃ and CHF₂), C₁₋₆alkoxyhalo (such as OCF₃ and OCHF₂), carboxyl, esters, cyano, nitro, amino, substituted amino, disubstituted amino, acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case N-heterocycles may also include but are not limited to C₁₋₆alkyl i.e. N—C₁₋₃alkyl, more preferably methyl particularly N-methyl.

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease. “Preventing” a disease or condition refers to prophylactic administering a composition to a subject who does not exhibit signs of a disease or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. F or a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions that will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

Particular examples of the presently disclosed compounds may include one or more asymmetric centers; thus these compounds can exist in different stereoisomeric forms. Accordingly, compounds and compositions may be provided as individual pure enantiomers or as stereoisomeric mixtures, including racemic mixtures. In certain embodiments the compounds disclosed herein are synthesized in or are purified to be in substantially enantiopure form, such as in a 90% enantiomeric excess, a 95% enantiomeric excess, a 97% enantiomeric excess or even in greater than a 99% enantiomeric excess, such as in enantiopure form.

The presently disclosed compounds can have at least one asymmetric center or geometric center, cis-trans center (C═C, C═N). All chiral, diasteromeric, racemic, meso, rotational and geometric isomers of the structures are intended unless otherwise specified. The compounds can be isolated as a single isomer or as mixture of isomers. All tautomers of the compounds are also considered part of the disclosure. The presently disclosed compounds also includes all isotopes of atoms present in the compounds, which can include, but are not limited to, deuterium, tritium, ¹⁸F, etc

### Overview

Disclosed herein are new pharmacological therapies that increase CYB5R3 activity, improve vascular function and limit inflammation by restoring the redox balance in CVD. In one embodiment, disclosed herein are compounds that include an alkyl chain containing a nitroalkene group conjugated to a quinone-containing moiety.

### Compounds, Pharmaceutical Compositions, and Methods of Use

Illustrative compounds include:
(A) Quinone-containing compounds such as:

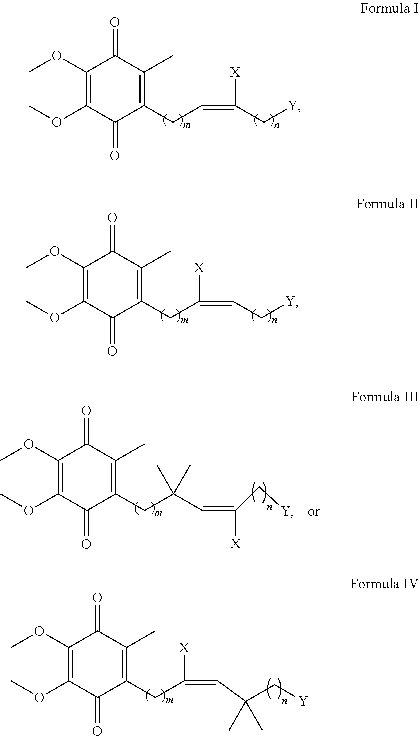

wherein X is an electron withdrawing group;
Y is a terminal group that inhibits enzymatic omega-end hydroxylation, oxidation and shortening of the alkyl chain to which Y is attached, or Y is H;
m is 1 to 10; and
n is 3 to 15.

In certain embodiments, X is nitro.

In certain embodiments, Y is H, dimethyl, trimethyl, cyclopropyl, or alkynyl.

In certain embodiments, m is 5 to 9.

In certain embodiments, n is 5 to 9.
(B) Napthoquinone-containing compounds such as

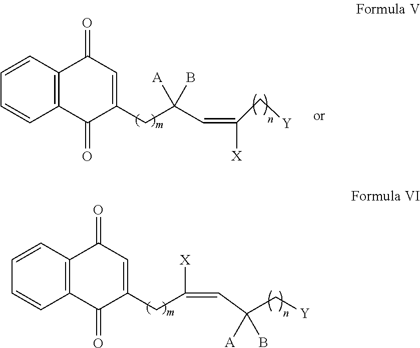

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

In certain embodiments, X is nitro.

In certain embodiments, Y is H, dimethyl, trimethyl, cyclopropyl, or alkynyl.

In certain embodiments, m is 5 to 9.

In certain embodiments, n is 5 to 9.
(C) Lapachol-containing compounds such as:

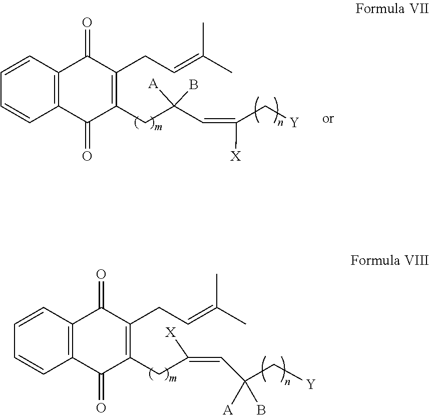

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

In certain embodiments, X is nitro.

In certain embodiments, Y is H, dimethyl, trimethyl, cyclopropyl, or alkynyl.

In certain embodiments, m is 5 to 9.

In certain embodiments, n is 5 to 9.
(D) Atovaquone-containing compounds such as:

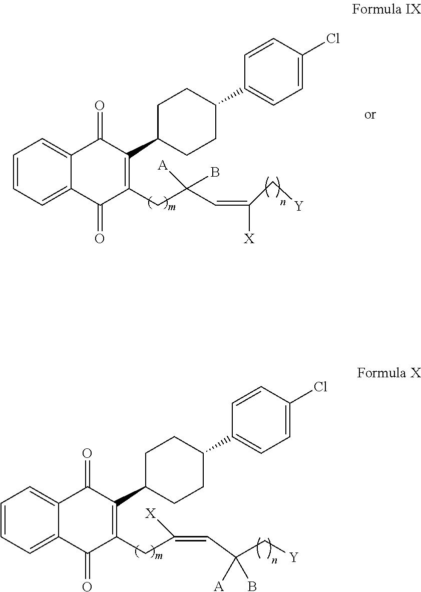

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

In certain embodiments, X is nitro.

In certain embodiments, Y is H, dimethyl, trimethyl, cyclopropyl, or alkynyl.

In certain embodiments, m is 5 to 9.

In certain embodiments, n is 5 to 9.
(E) Parvaquone-containing compounds such as:

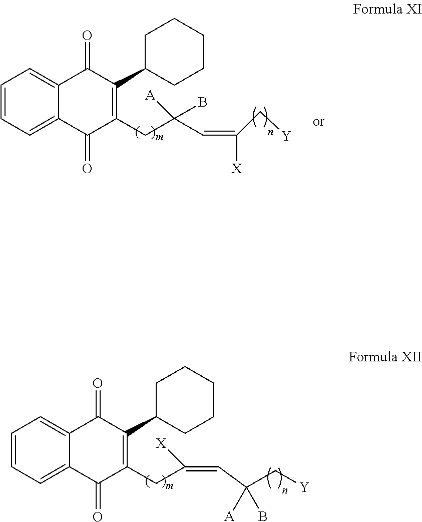

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

In certain embodiments, X is nitro.

In certain embodiments, Y is H, dimethyl, trimethyl, cyclopropyl, or alkynyl.

In certain embodiments, m is 5 to 9.

In certain embodiments, n is 5 to 9.
(F) Benzylmenadione-containing compounds such as

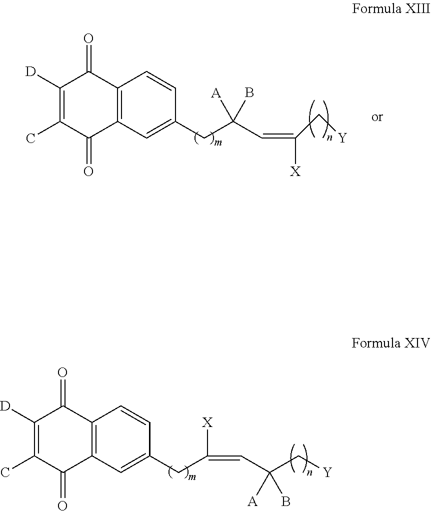

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl; C and D are each independently H or CH₃;
m is 1 to 10; and
n is 3 to 15.

In certain embodiments, X is nitro.

In certain embodiments, Y is H, dimethyl, trimethyl, cyclopropyl, or alkynyl.

In certain embodiments, m is 5 to 9.

In certain embodiments, n is 5 to 9.
(G) Isoprenoid-containing compound such as:

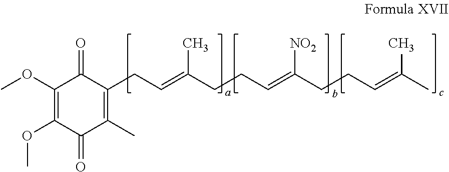

wherein a is 0 to 9, b is 1, and c is 0 to 9.
(H) Benzoquinone-containing compounds such as

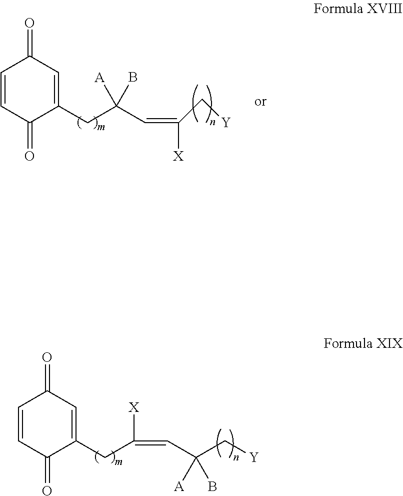

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

In certain embodiments, X is nitro.

In certain embodiments, Y is H, dimethyl, trimethyl, cyclopropyl, or alkynyl.

In certain embodiments, m is 5 to 9.

In certain embodiments, n is 5 to 9.
(I) Anthraquinone-containing compounds such as

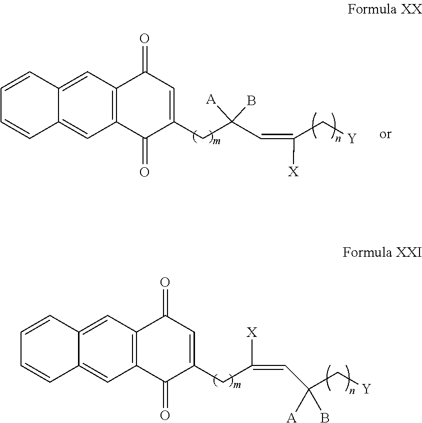

wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

In certain embodiments, X is nitro.

In certain embodiments, Y is H, dimethyl, trimethyl, cyclopropyl, or alkynyl.

In certain embodiments, m is 5 to 9.

In certain embodiments, n is 5 to 9.

The term “electron-withdrawing group” is recognized in the art and denotes the tendency of a substituent to attract valence electrons from neighboring atoms, i.e., the substituent is electronegative with respect to neighboring atoms. Embodiments encompass any known electron withdrawing group. For example, electron-withdrawing groups may include, but are not limited to, aldehyde (—COH), acyl (—COR), carboxylic acid (—COOH), ester (—COOR), halides (—Cl, F, —Br, etc.), fluoromethyl (—CF₃), fluoroalkyl (—CFₙH₂₋ₙR), cyano (—CN), sulfoxide (—SOR), sulfonyl (—SO₂R), sulfonate (SO₃R), 1°, 2° and 3° ammonium (—NR₃⁺), and nitro (—NO₂) where each R may, independently, be hydrogen, methyl, C₂ to C₆ alkyl, alkenyl, or alkynyl. In some embodiments, the electron withdrawing group may be a strong electron withdrawing group having a 6 of at least about 0.2, and in certain embodiments, the electron withdrawing group may form a dipole. For example, in particular embodiments, the electron withdrawing group may be a nitro, ammonium or sulfonyl.

In certain embodiments, the compounds are:

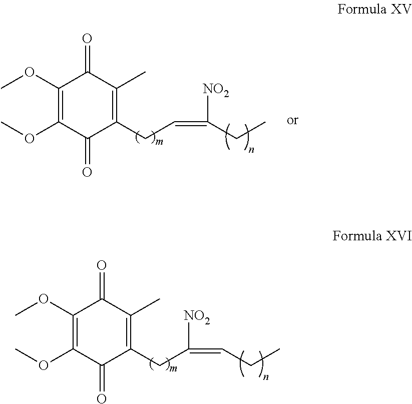

wherein m is 1 to 10 and n is 3 to 15.

In certain embodiments, m is 5 to 9.

In certain embodiments, n is 5 to 9.

Illustrative compounds include:

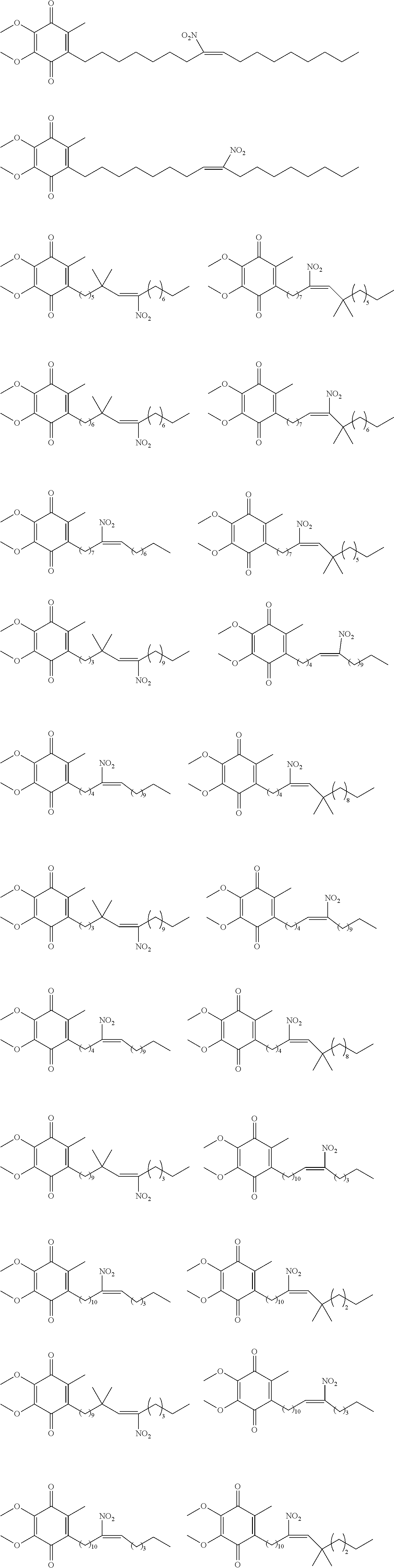

In certain embodiments, the compounds disclosed herein may be used for treating or preventing diseases or conditions associated with a redox imbalance and/or inflammatory signaling. In various embodiments, the disease or condition may be, but may not be limited to, arterial stiffness, systemic and pulmonary hypertension, sickle cell disease, heart failure with reduced ejection fraction, heart failure with preserved ejection fraction, arterial stenosis, vascular aneurysm, obesity, neurodegenerative disorders, skin disorders, arthritis, vasculitis, burns, autoimmune disease, autoinflammatory disease, lupus, Lyme's disease, gout, sepsis, hyperthermia, ulcers, enterocolitis, osteoporosis, viral or bacterial infections, cytomegalovirus, periodontal disease, glomerulonephritis, sarcoidosis, lung disease, chronic lung injury, respiratory distress, lung inflammation, fibrosis of the lung, asthma, acquired respiratory distress syndrome, tobacco induced lung disease, granuloma formation, fibrosis of the liver, graft vs. host disease, postsurgical inflammation, coronary and peripheral vessel restenosis following angioplasty, stent placement or bypass graft, acute and chronic leukemia, B lymphocyte leukemia, neoplastic diseases, arteriosclerosis, atherosclerosis, myocardial inflammation and fibrosis, psoriasis, immunodeficiency, disseminated intravascular coagulation, systemic sclerosis, amyotrophic lateral sclerosis, multiple sclerosis, Parkinson's disease, Alzheimer's disease, encephalomyelitis, edema, inflammatory bowel disease, hyper IgE syndrome, cancer metastasis or growth, adoptive immune therapy, reperfusion syndrome, radiation burns, or alopecia.

For example, in certain embodiments, the compounds disclosed may be used for treating or preventing cardiovascular disease including arterial stiffness, systemic and pulmonary hypertension, heart failure with reduced ejection fraction, heart failure with preserved ejection fraction, renal stenosis, glomerulosclerosis, atherosclerosis, arteriosclerosis, thrombosis, or sickle cell disease.

In certain embodiments, the compounds disclosed herein may enhance CYB5R3 activity.

In certain embodiments, the compounds disclosed herein may be used for treating or preventing diseases or conditions associated with modulation of vascular function/blood pressure regulation.

In certain embodiments, the compounds disclosed herein may be used for treating or preventing diseases or conditions associated with sickle cell disease-induced vascular injury impacts other organ systems such as the kidney or lung.

In certain embodiments, the compounds disclosed herein may inhibit inflammatory conditions or diseases, particularly those associated with pro-inflammatory genes IL-Iβ, VCAM-1, ICAM-1 CCL5, CCL20 and CXCL10.

In certain embodiments, the compounds disclosed herein may decrease mean arterial blood pressure in a subject.

In certain embodiments, the compounds disclosed herein may decrease pulse pressure in a subject.

In certain embodiments, the compounds disclosed herein may reverse endothelial cell dysfunction.

In certain embodiments, the subject is in need of, or has been recognized as being in need of, treatment with an anti-CVD agent or anti-inflammatory agent. The subject may be selected as being amenable to treatment with an anti-CVD agent or anti-inflammatory agent.

In some embodiments, the methods disclosed herein involve administering to a subject in need of treatment a pharmaceutical composition, for example a composition that includes a pharmaceutically acceptable carrier and a therapeutically effective amount of one or more of the compounds disclosed herein. The compounds may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

In some embodiments, one or more of the disclosed compounds (including compounds linked to a detectable label or cargo moiety) are mixed or combined with a suitable pharmaceutically acceptable carrier to prepare a pharmaceutical composition. Pharmaceutical carriers or vehicles suitable for administration of the compounds provided herein include any such carriers known to be suitable for the particular mode of administration. Remington: The Science and Practice of Pharmacy, The University of the Sciences in Philadelphia, Editor, Lippincott, Williams, & Wilkins, Philadelphia, PA, 21St Edition (2005), describes exemplary compositions and formulations suitable for pharmaceutical delivery of the compounds disclosed herein. In addition, the compounds may be formulated as the sole pharmaceutically active ingredient in the composition or may be combined with other active ingredients.

Upon mixing or addition of the compound(s) to a pharmaceutically acceptable carrier, the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions. The disclosed compounds may also be prepared with carriers that protect them against rapid elimination from the body, such as time-release formulations or coatings. Such carriers include controlled release formulations, such as, but not limited to, microencapsulated delivery systems.

The disclosed compounds and/or compositions can be enclosed in multiple or single dose containers. The compounds and/or compositions can also be provided in kits, for example, including component parts that can be assembled for use. For example, one or more of the disclosed compounds may be provided in a lyophilized form and a suitable diluent may be provided as separated components for combination prior to use.

The active compound is included in the pharmaceutically acceptable carrier in an amount sufficient to exert a therapeutically useful effect in the absence of undesirable side effects on the subject treated. A therapeutically effective concentration may be determined empirically by testing the compounds in known in vitro and in vivo model systems for the treated disorder. In some examples, a therapeutically effective amount of the compound is an amount that lessens or ameliorates at least one symptom of the disorder for which the compound is administered. Typically, the compositions are formulated for single dosage administration. The concentration of active compound in the drug composition will depend on absorption, inactivation, and excretion rates of the active compound, the dosage schedule, and amount administered as well as other factors known to those of skill in the art.

In some examples, about 0.1 mg to 1000 mg of a disclosed compound, a mixture of such compounds, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. The pharmaceutical compositions may be in a dosage unit form such as an injectable fluid, an oral delivery fluid (e.g., a solution or suspension), a nasal delivery fluid (e.g., for delivery as an aerosol or vapor), a semisolid form (e.g., a topical cream), or a solid form such as powder, pill, tablet, or capsule forms.

In some examples, the compositions are formulated in a unit dosage form, each dosage containing from about 1 mg to about 1000 mg (for example, about 2 mg to about 500 mg, about 5 mg to 50 mg, about 10 mg to 100 mg, or about 25 mg to 75 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, or more of the disclosed compound(s).

The disclosed compounds or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data. It is to be noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants. If oral administration is desired, the compound is typically provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily. In some examples, an oral dosage form is administered to the subject 1, 2, 3, 4, or more times daily. In additional examples, the compounds can be administered orally to humans in a dosage range of 1 to 1000 mg/kg body weight in single or divided doses. One illustrative dosage range is 0.1 to 200 mg/kg body weight orally (such as 0.5 to 100 mg/kg body weight orally) in single or divided doses. For oral administration, the compositions may be provided in the form of tablets containing about 1 to 1000 milligrams of the active ingredient, particularly 1, 5, 10, 15, 20, 25, 50, 75, 100, 150, 200, 250, 300, 400, 500, 600, 750, 800, 900, or 1000 milligrams of the active ingredient. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The compounds can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The compounds can also be administered sublingually. When given sublingually, the compounds should be given one to four times daily in the amounts described above for IM administration.

The compounds can also be administered intranasally. When given by this route, the appropriate dosage forms are a nasal spray or dry powder. The dosage of the compounds for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The compounds can be administered intrathecally. When given by this route, the appropriate dosage form can be a parenteral dosage form. The dosage of the compounds for intrathecal administration is the amount described above for IM administration.

The compounds can be administered topically. When given by this route, the appropriate dosage form is a cream, ointment, or patch. When administered topically, an illustrative dosage is from about 0.5 mg/day to about 200 mg/day. Because the amount that can be delivered by a patch is limited, two or more patches may be used.

The compounds can be administered rectally by suppository. When administered by suppository, an illustrative therapeutically effective amount may range from about 0.5 mg to about 500 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug. It should be apparent to one skilled in the art that the exact dosage and frequency of administration will depend on the particular compounds administered, the particular condition being treated, the severity of the condition being treated, the age, weight, general physical condition of the particular subject, and other medication the individual may be taking as is well known to administering physicians or other clinicians who are skilled in therapy of retroviral infections, diseases, and associated disorders.

### Examples

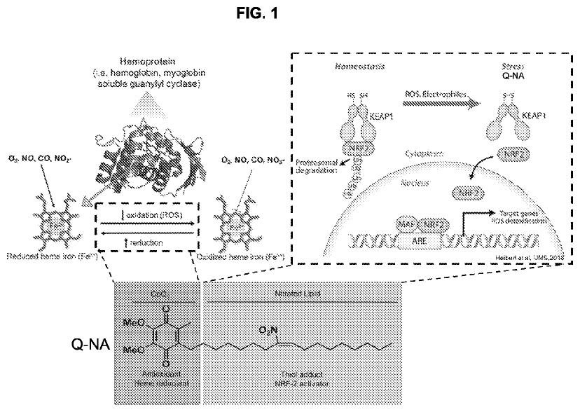

A modified nitrated fatty acid that is conjugated to CoQ₀, termed Q-NA was synthesized (FIG. 1).

Q-NA has pleotropic action within cells. First, the CoQ group reduces heme iron (a target of oxidants), reduces oxidant formation and increases CYB5R3 activity. Second, the conjugation of the CoQ₀ to a nitroalkene-containing substituent provides the added benefit of activating the antioxidant pathway through NRF-2. In addition to its redox properties, Q-NA is a lipid-soluble hydrophobic species that can be rapidly and efficiently taken up by cells, especially by endothelial cell and cardiomyocytes as lipids are a major source of their energy.

### Methods:

Synthesis of nitroalkene-quinone compounds—A recent report by Wang et al, Tetrahedron. 2014; 70(47):9029-32, described a simple and straightforward method for the direct conjugation of alkyl carboxylic acids and ubiquinone via a radical decarboxylation coupling. Commercially available Q₀ (2,3-Dimethoxy-5-methyl-p-benzoquinone) was reacted with silver nitrate and nitro-oleic acid (mixture of 9-, 10-nitro octadece-9-noic acid) or oleic acid (octadece-9-noic acid) in the presence of sodium persulfate in an acetonitrile-water solution and then heated for 2-3 hours. The resulting solution was extracted and washed, followed by removal of solvent under reduced pressure and purification of the crude product by column chromatography. The final product was identified by ¹H, ¹³C nuclear resonance imaging.

Synthesis of other compounds:

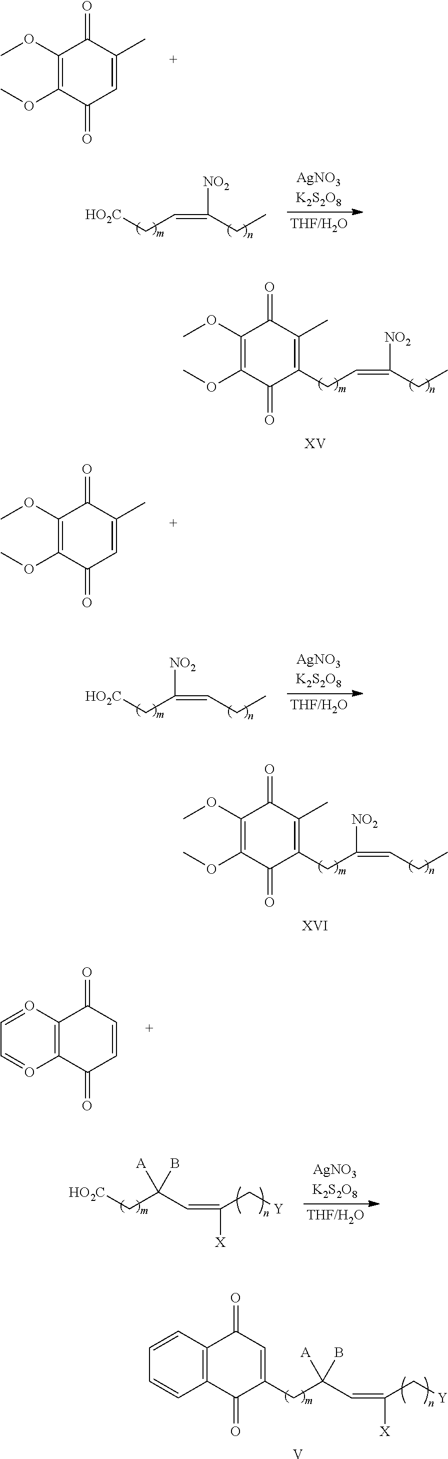

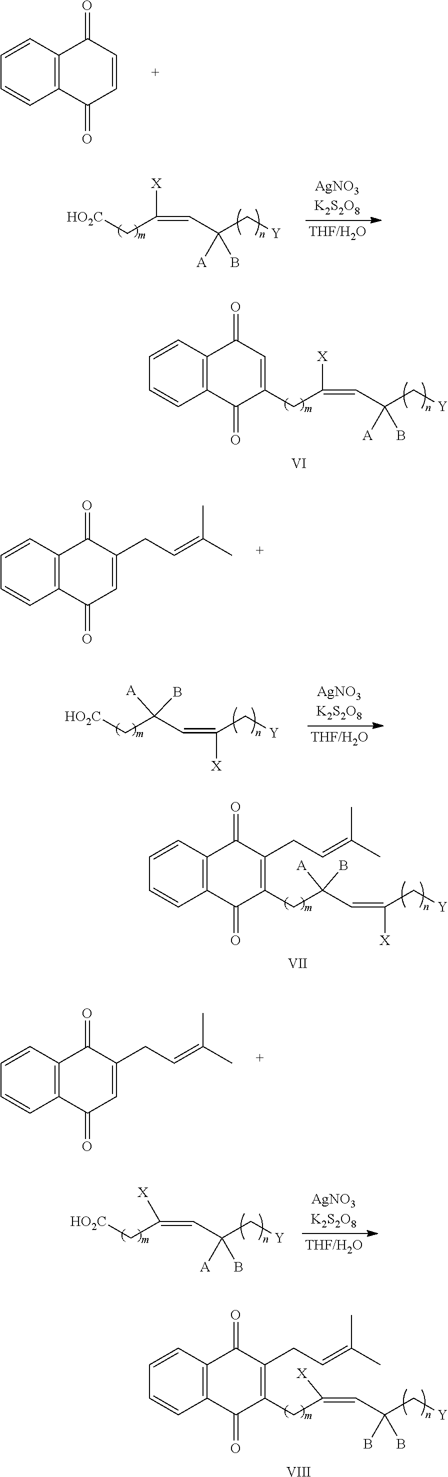

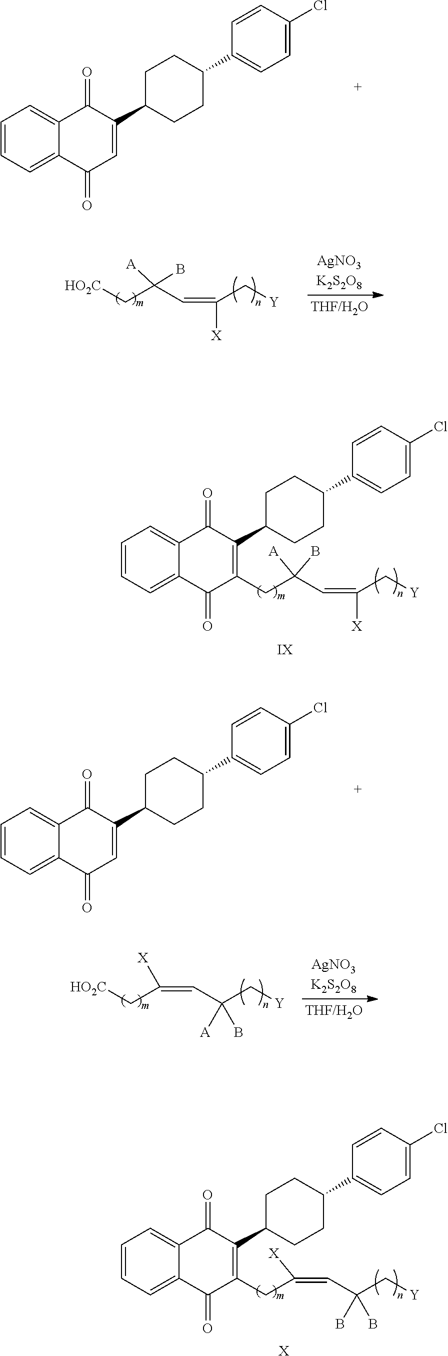

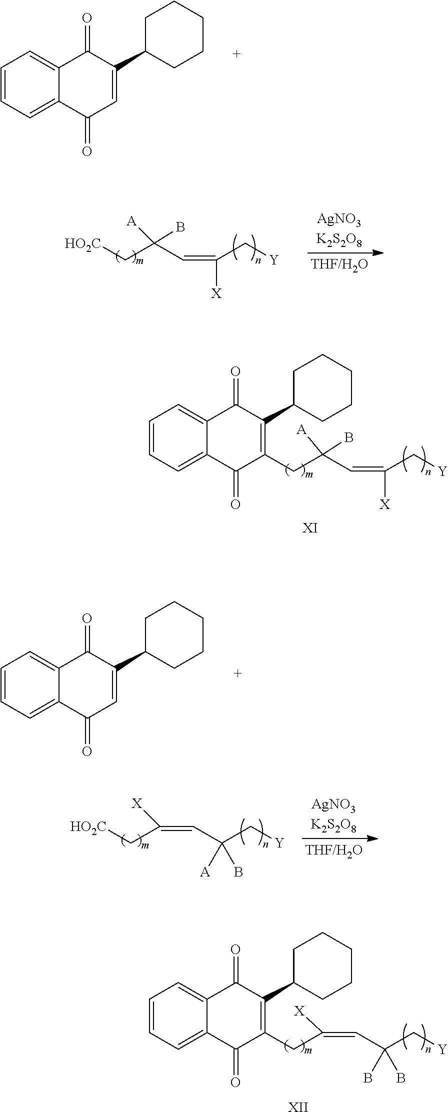

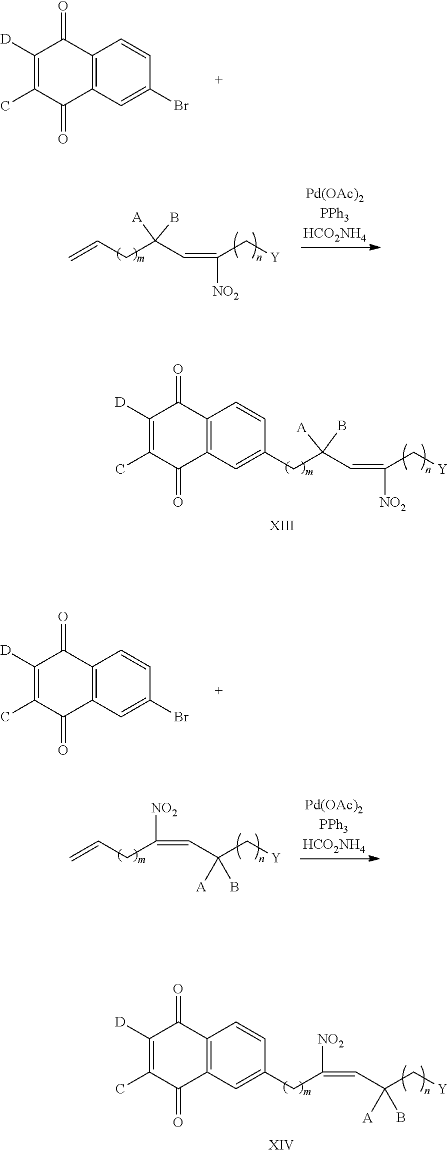

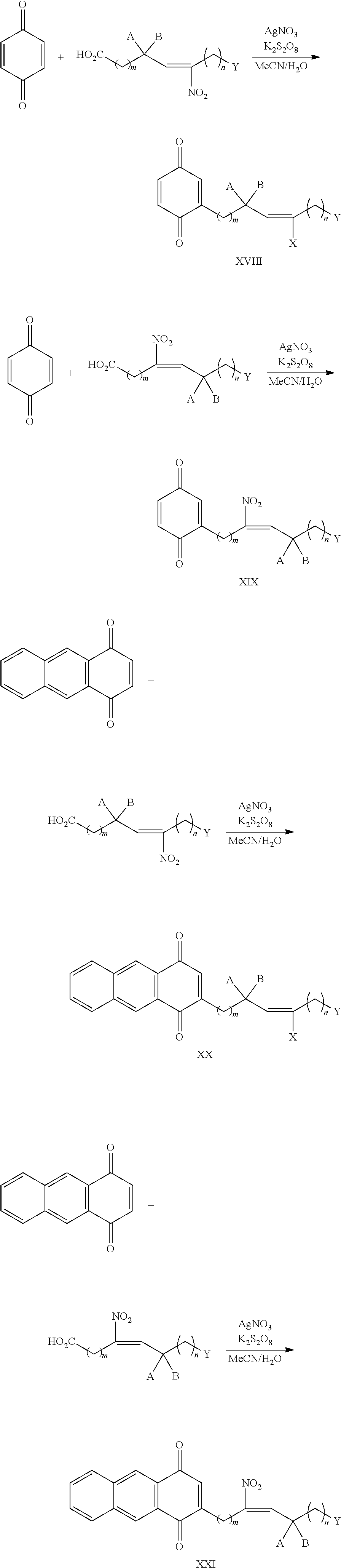

### Results:

### Q-NA Enhances CYB5R3 Activity.

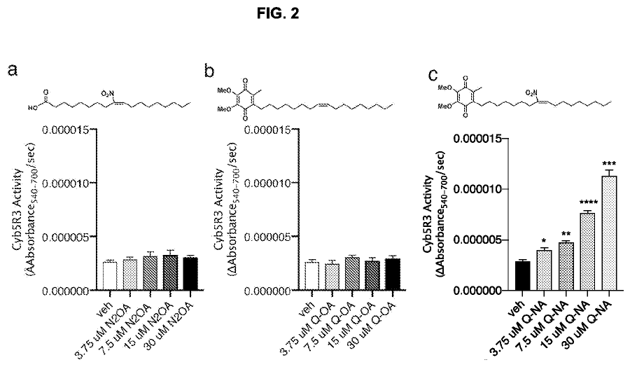

Recombinant CYB5R3 was incubated with nitro-oleic acid, CoQ-linked oleic acid or Q-NA along with oxidized myoglobin. NADH stimulation initiated the reaction and absorbance changes were monitored which is indicative of myoglobin reduction. Q-NA increased myoglobin heme reduction whereas the other compounds had no effect (FIG. 2). Q-NA alone had no effect on myoglobin reduction. This indicates that Q-NA enhances CYB5R3 activity.

Gene expression profiling reveals differential and unique gene expression profiles in human aortic endothelial cells exposed to Q-NA, as opposed to oleic acid (OA), nitro-oleic acid (NO₂—OA) and quinone-conjugated oleic acid (Q-OA).

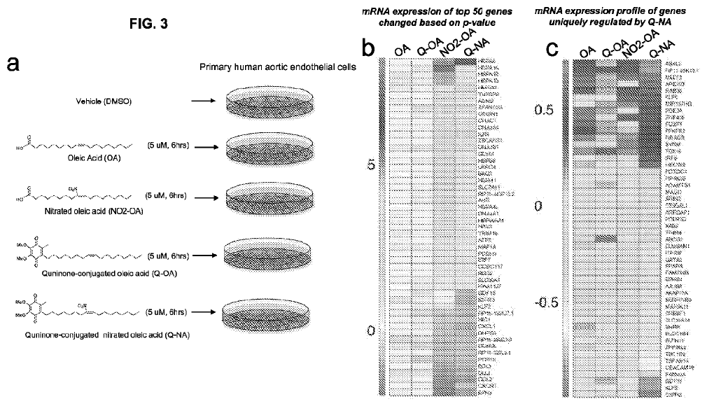

To assess the impact of Q-NA on gene expression, we conducted an RNAseq analysis on cultured human aortic endothelial cells (HAECs). To do so, HAECs were incubated with vehicle (DMSO), 5 μM oleic acid (OA), 5 μM nitrated oleic acid (NO₂—OA), 5 μM quinone-conjugated oleic acid (Q-OA) or 5 μM quinone-conjugated nitro oleic acid (Q-NA) for 6 hours. Following treatment, HAECs were lysed and mRNA was isolated is subjected to RNAseq analysis. Heat maps show changes in the top 50 genes sorted based on p-value (middle heat map) or changes in gene expression uniquely regulated by Q-NA (right heat map) (FIG. 3). These results show that Q-NA modulates gene expression profiles similarly and uniquely compared to other nitrated and non-nitrated lipids.

### Q-NA Tempers Inflammatory Signaling.

To test if Q-NA exerts anti-inflammatory actions, we co-treated HAECs with vehicle (DMSO), 5 μM of Q-OA, N₂OA or Q-NA with and without 1 ug/mL of lipopolysaccharide to induce inflammation.

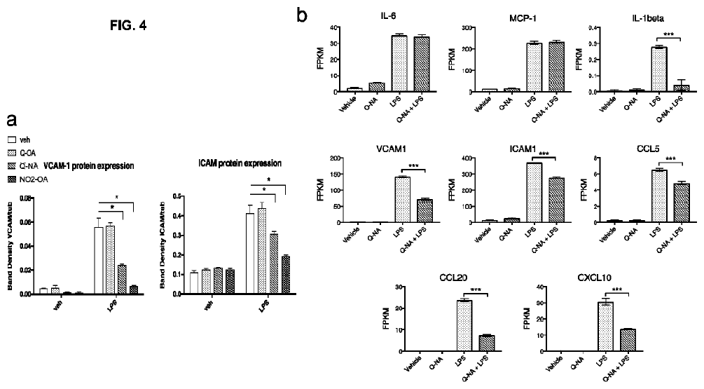

Results shown in FIG. 4a demonstrate that Q-NA tempers the induction of pro-inflammatory proteins VCAM-1 and ICAM-1. Together these results demonstrate that Q-NA exerts anti-inflammatory actions. To determine if Q-NA impacted transcription of pro-inflammatory genes, we conducted an RNAseq analysis on human aortic endothelial cells treated with vehicle (DMSO), 5 μM of Q-OA, 1 ug/mL of lipopolysaccharide and 5 μM of Q-OA+1 ug/mL of lipopolysaccharide. We found that Q-NA suppressed IL-IP, VCAM-1, ICAM-1 CCL5, CCL20 and CXCL10 mRNA expression but not IL-6 or MCP-1 expression (FIG. 4b).

### Q-NA Decreases Mean Arterial Blood Pressure, Pulse Pressure and Improves Endothelial Function in a 2 Kidney, 1 Renal Artery Clip Mouse Model.

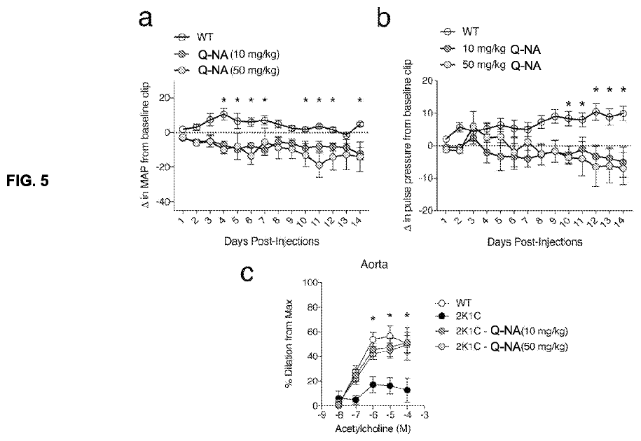

We have recently established a high salt (4%), 2 kidney, 1 renal artery clip mouse model that leads to hypertension and aortic stiffness in C57Bl/6 mice. Using this model, we implanted radiotelemetry units to measure blood pressure. After two weeks of establishing a hypertensive phenotype, we injected Q-NA via intraperitoneal injection every other day for two weeks with 10 or 50 mg/kg of Q-NA. Results shown in (FIG. 5a) show that both high and low doses of Q-NA decrease blood pressure and pulse pressure (FIG. 5b) (an indicator of aortic stiffness). After two weeks animals were sacrificed and aortic rings were subjected to vascular function tests. Results shown in FIG. 5c demonstrate that both low and high dose of Q-NA completely reverse endothelial dysfunction. These results demonstrate that Q-NA decreases mean arterial and pulse pressure and exerts vascular protective actions in a 2 kidney, 1 clip mouse model.

### Q-NA Reverses Anemia and Improves Endothelial and Smooth Muscle Cell Function in Sickle Cell Disease Mice.

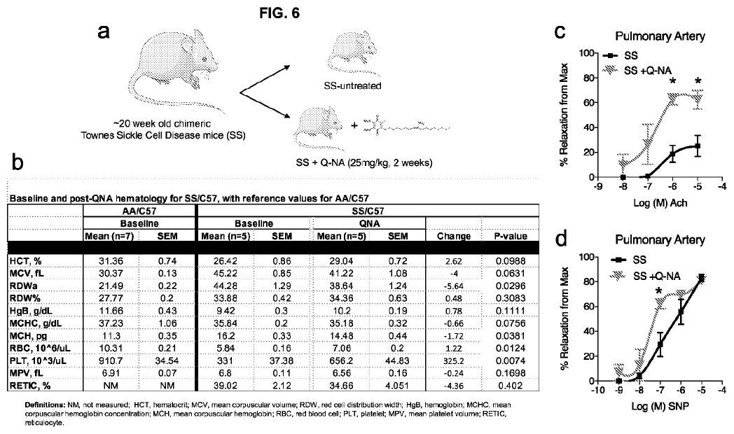

To determine whether Q-NA impacts anemia and vascular function in sickle cell disease (SCD), we treated chimeric SCD mice with 25 mg/kg of Q-NA every other day for two weeks (FIG. 6a). Baseline, hematology from a normal C57Bl/6 mice (AA/C57) is shown in FIG. 6b, left column. Baseline hematology from SCD mice prior to Q-NA treatment is shown in FIG. 6b middle column and following 2 weeks of Q-NA shown in right column. An increase in red blood cells and hemoglobin indicate reversal of anemia. We also found significant improvement in endothelial (FIG. 6c) and smooth muscle (FIG. 6d) function from SCD mice treated with 2 weeks of Q-NA.

### Modified Quinone-Derivatives Impact Gene Expression Profiles.

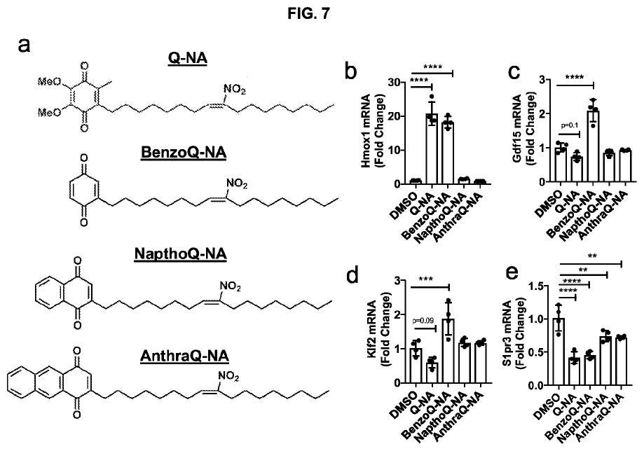

To test if modified quinone derivative impacted gene expression profiles of Hmox1, GDF-15, KLF-2 and Slpr3, we synthesized benzoQ-NA, napthoQ-NA, and anthraQ-NA. Structure-activity relationships indicate that quinone-derivatives differentially modulate gene expression profiles of Hmox1, GDF-15, KLF-2 and S1pr3 (FIG. 7).

Q-NA requires CYB5R3 to regulate Ribosomal Protein S and L (RPS/RPL) gene expression with and without lipopolysaccharide.

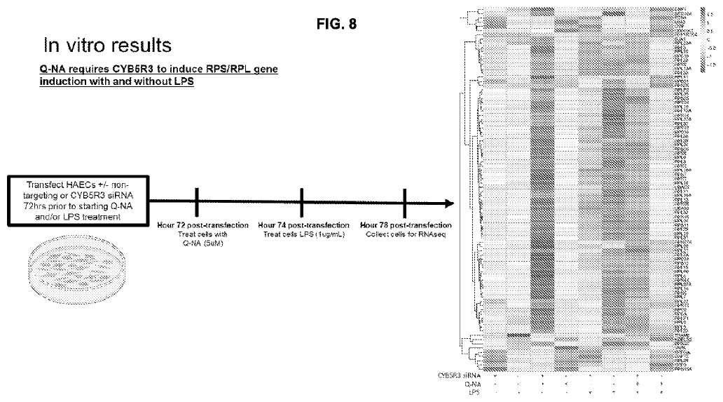

To examine whether Q-NA requires CYB5R3 expression to regulate gene expression in the presence or absence of lipopolysaccharide, human aortic endothelial cells were transfected with non-targeting (nt-RNA) or CYB5R3 siRNA for 72 hrs. Cells were then treated with 5 μM Q-NA. After 2 hours cells, treated with 1 ug/mL of lipopolysaccharide for 4 hours. Cells were then lysed and RNA was collected for bulk RNA sequencing analysis. We found that CYB5R3 expression is required for Q-NA to modulate RPS/RPL gene family expression in the presence and absence of lipopolysaccharide (FIG. 8).

### NO₂—OA and Q-NA Stimulate Divergent Effect on RPS/RPL Gene Transcription

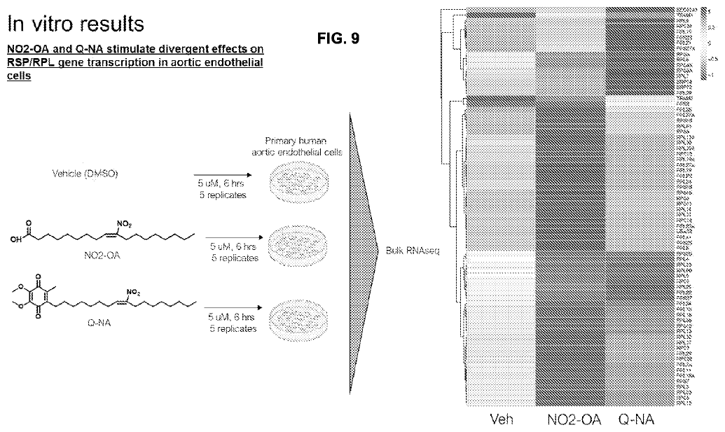

Human aortic endothelial cells were stimulated with vehicle (dimethylsulfoxide, (DMSO)), 5 μM of NO₂—OA, or 5 μM Q-NA. Following 6 hours of stimulation, cells were lysed and RNA sequencing was performed. Results show that NO₂—OA caused opposing effects on RPS/RPL gene expression compared to Q-NA treatment (FIG. 9).

Together these results demonstrate that the novel quinone-conjugated nitroalkene-containing compound 1) increases CYB5R3 activity, 2) uniquely activates endothelial gene programs, 3) tempers inflammation, 4) decreases mean arterial and pulse pressure and reverses endothelial cell dysfunction in a 2 kidney, 1 renal artery clip mouse model, 5) Q-NA reverses anemia and endothelial/smooth muscle dysfunction in sickle cell disease, 6) modified quinone derivatives impact gene expression profiles of Hmox1, GDF-15, KLF-2 and Slpr3, 7) Q-NA requires CYB5R3 to regulate RPS/RPL gene expression with and without lipopolysaccharide and 8) NO₂—OA and Q-NA stimulate divergent effect on RPS/RPL gene transcription. These results implicate that Q-NA provides protection against high blood pressure, aortic stiffness, and sickle cell disease through expression of CYB5R3.

In view of the many possible embodiments to which the principles of the disclosed invention may be applied, it should be recognized that the illustrated embodiments are only preferred examples of the invention and should not be taken as limiting the scope of the invention.

1. A compound, or a pharmaceutically acceptable salt thereof, of: 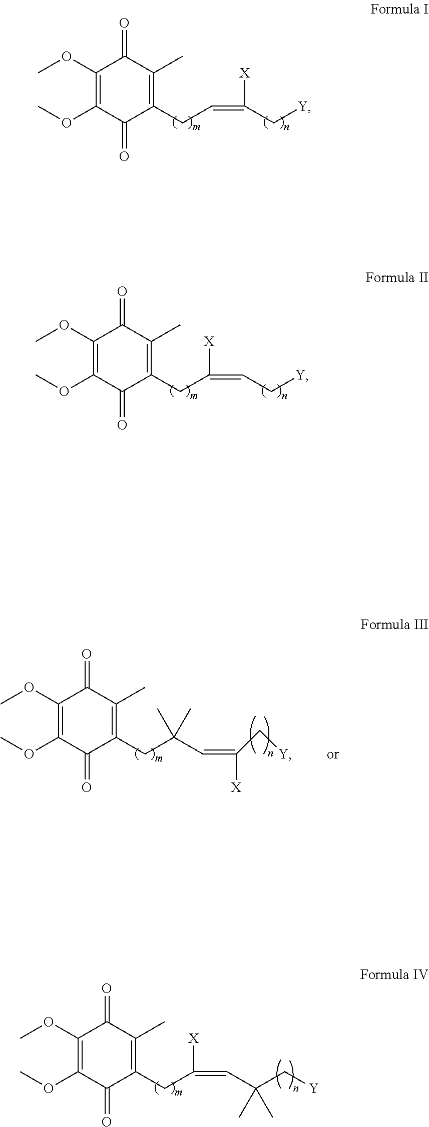
wherein X is an electron withdrawing group;
Y is a terminal group that inhibits enzymatic omega-end hydroxylation, oxidation and shortening of the alkyl chain to which Y is attached, or Y is H;
m is 1 to 10; and
n is 3 to 15.

2. The compound of claim 1, wherein the compound is:

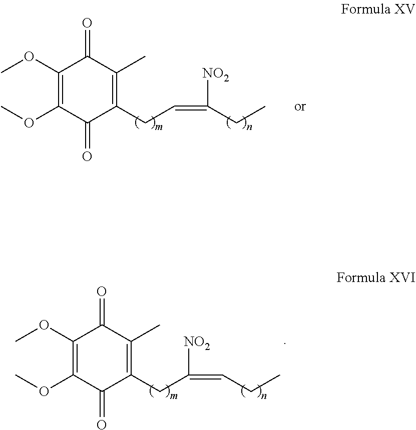

3. The compound of claim 1, wherein the compound is:

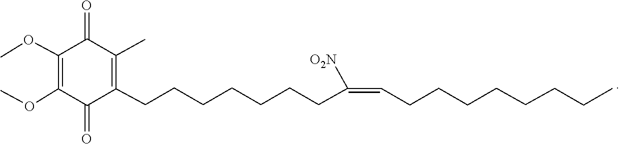 [SMILES: CCCCCCCC/C=C(\CCCCCCCC1=C(C)C(=O)C(OC)=C(OC)C1=O)[N+](=O)[O-]]

4. The compound of claim 1, wherein the compound is:

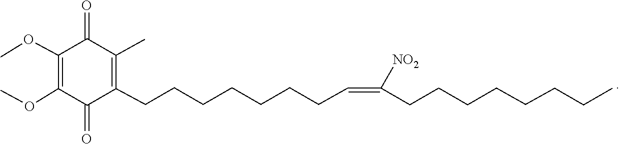 [SMILES: CCCCCCCC/C(=C\CCCCCCCC1=C(C)C(=O)C(OC)=C(OC)C1=O)[N+](=O)[O-]]

5. A compound, or a pharmaceutically acceptable salt thereof, of: 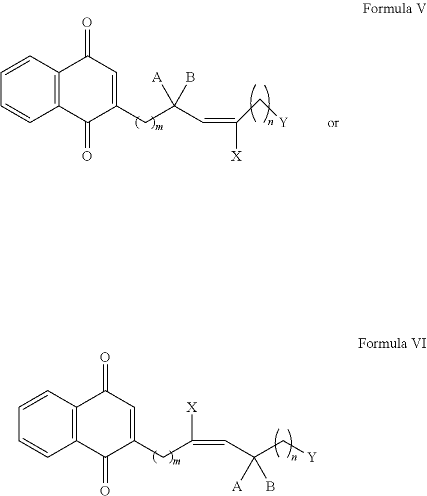
wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

6. A compound, or a pharmaceutically acceptable salt thereof, of: 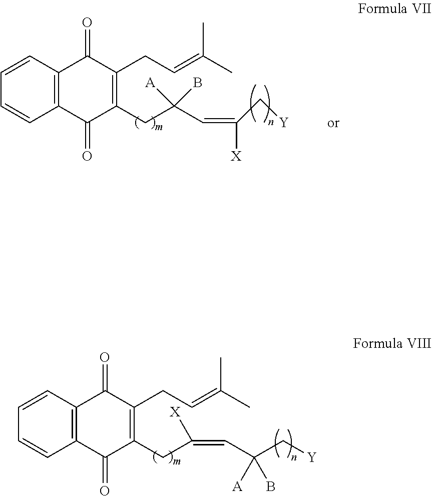
wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

7. A compound, or a pharmaceutically acceptable salt thereof, of: 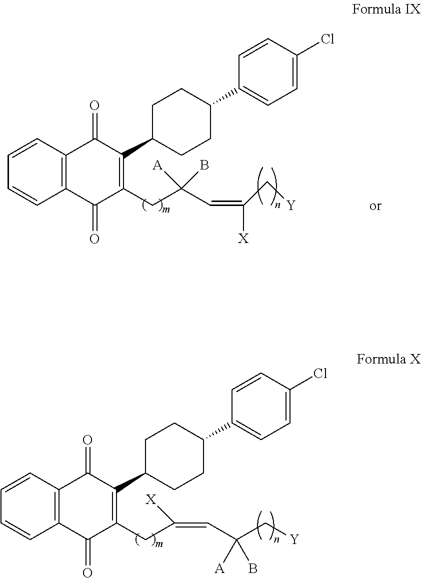
wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

8. A compound, or a pharmaceutically acceptable salt thereof, of: 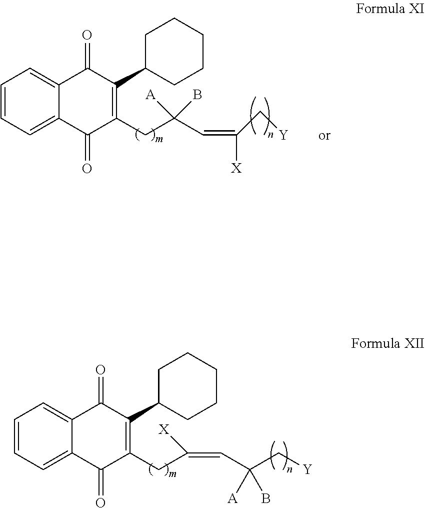
wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

9. A compound, or a pharmaceutically acceptable salt thereof, of: 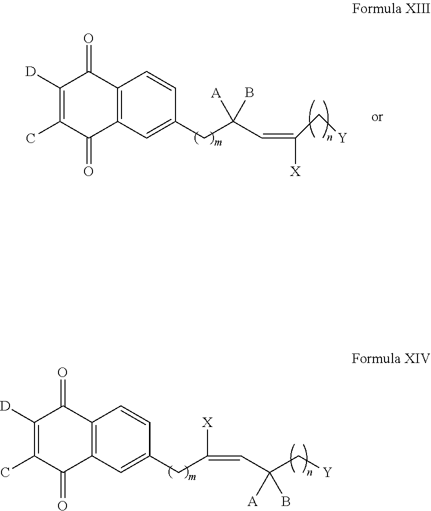
wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

10. A compound, or a pharmaceutically acceptable salt thereof, of: 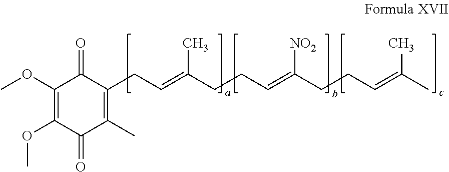
wherein a is 0 to 9, b is 1, and c is 0 to 9.

11. A compound, or a pharmaceutically acceptable salt thereof, of: 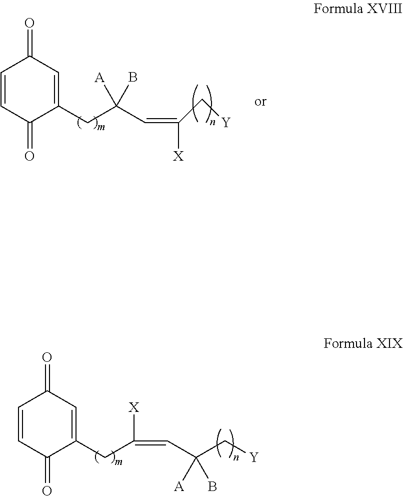
wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

12. A compound, or a pharmaceutically acceptable salt thereof, of: 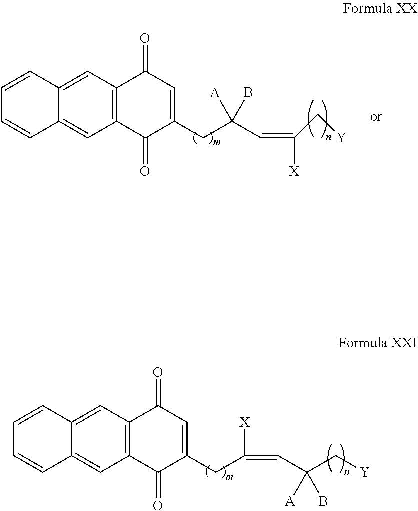
wherein X is an electron withdrawing group;
Y is a terminal group that inhibits enzymatic omega-end hydroxylation, oxidation and shortening of the alkyl chain to which Y is attached, or Y is H;
A and B are each independently H or CH₃ or A and B together form a cyclopropyl;
m is 1 to 10; and
n is 3 to 15.

13. The compound of claim 1, wherein the electron-withdrawing group is aldehyde (—COH), acyl (—COR), carboxylic acid (—COOH), ester (—COOR), halides (—Cl, F, —Br, etc.), fluoromethyl (—CF₃), fluoroalkyl, cyano (—CN), sulfoxide (—SOR), sulfonyl (—SO₂R), sulfonate (SO₃R), 1°, 2° and 3° ammonium (—NR₃⁺), or nitro (—NO₂) where each R may, independently, be hydrogen, methyl, C₂ to C₆ alkyl, alkenyl, or alkynyl.

14. The compound of claim 1, wherein the electron-withdrawing group is nitro.

15. The compound of claim 1, wherein Y is H, dimethyl, trimethyl, cyclopropyl, or alkynyl.

16. A method comprising administering a therapeutically effective amount of a compound of claim 1 to a subject having, suspected of having, or at risk of developing, cardiovascular disease.

17. A method of treating cardiovascular disease in a subject, comprising administering a therapeutically effective amount of a compound of claim 1 to the subject, thereby treating the cardiovascular disease.

18. A method of treating cardiovascular disease in a subject, comprising administering a therapeutically effective amount of a compound comprising an alkyl chain containing a nitroalkene group, wherein the alkyl chain containing a nitroalkene group is conjugated to a quinone-containing moiety, thereby treating the cardiovascular disease.

19. A method of treating a disease or condition associated with a redox imbalance in a subject, comprising administering a therapeutically effective amount of a compound of claim 1 to the subject, thereby treating the disease or condition associated with a redox imbalance.

20. A method comprising administering to a subject a compound of claim 1, thereby enhancing CYB5R3 activity in the subject.

21. A method for inhibiting an inflammatory condition or disease associated with pro-inflammatory genes IL-1β, VCAM-1, ICAM-1 CCL5, CCL20 or CXCL10 in a subject, comprising administering a therapeutically effective amount of a compound of claim 1 to the subject, thereby treating the inflammatory condition or disease.

22. A method comprising administering to a subject a compound of claim 1, thereby decreasing mean arterial blood pressure in the subject.

23. A method comprising administering to a subject a compound of claim 1, thereby decreasing pulse pressure in the subject.

24. A method comprising administering to a subject a compound of claim 1, thereby reversing endothelial cell dysfunction in the subject.

25. A method of treating sickle cell disease in a subject, comprising administering a therapeutically effective amount of a compound of claim 1 to the subject, thereby treating the sickle cell disease.

26. A method of treating a disease or condition in a subject, comprising administering a therapeutically effective amount of a compound of claim 1 to the subject, thereby treating the disease or condition, wherein the disease or condition is arterial stiffness, systemic and pulmonary hypertension, sickle cell disease, heart failure with reduced ejection fraction, heart failure with preserved ejection fraction, arterial stenosis, vascular aneurysm, obesity, neurodegenerative disorders, skin disorders, arthritis, vasculitis, burns, autoimmune disease, autoinflammatory disease, lupus, Lyme's disease, gout, sepsis, hyperthermia, ulcers, enterocolitis, osteoporosis, viral or bacterial infections, cytomegalovirus, periodontal disease, glomerulonephritis, sarcoidosis, lung disease, chronic lung injury, respiratory distress, lung inflammation, fibrosis of the lung, asthma, acquired respiratory distress syndrome, tobacco induced lung disease, granuloma formation, fibrosis of the liver, graft vs. host disease, postsurgical inflammation, coronary and peripheral vessel restenosis following angioplasty, stent placement or bypass graft, acute and chronic leukemia, B lymphocyte leukemia, neoplastic diseases, arteriosclerosis, atherosclerosis, myocardial inflammation and fibrosis, psoriasis, immunodeficiency, disseminated intravascular coagulation, systemic sclerosis, amyotrophic lateral sclerosis, multiple sclerosis, Parkinson's disease, Alzheimer's disease, encephalomyelitis, edema, inflammatory bowel disease, hyper IgE syndrome, cancer metastasis or growth, adoptive immune therapy, reperfusion syndrome, radiation burns, or alopecia.
